(19) United States
(12) Patent Application Publication
(10) Pub. No.: US 2026/0104381 A1
(43) Pub. Date: Apr. 16, 2026

(54) ELECTRODEPOSITED METAL MODIFIED LASER SCRIBED GRAPHENE ELECTRODE AND METHOD

(71) Applicant: KING ABDULLAH UNIVERSITY OF SCIENCE AND TECHNOLOGY, Thuwal (SA)

(72) Inventors: Sakandar RAUF, Thuwal (SA); Abdellatif AIT LAHCEN, Thuwal (SA); Abdulrahman ALJEDAIBI, Thuwal (SA); Khaled Nabil SALAMA, Thuwal (SA)

(21) Appl. No.: 19/407,472

(22) Filed: Dec. 3, 2025

Related U.S. Application Data
(62) Division of application No. 17/924,418, now abandoned.
(60) Provisional application No. 63/024,756, filed on May 14, 2020.

(51) Int. Cl. G01N 27/327; C25D 3/48; C25D 5/56; C25D 7/00; C25D 9/02; G01N 33/53; G01N 33/543

(52) U.S. Cl. CPC G01N 27/3278; C25D 3/48; C25D 5/56; C25D 7/00; C25D 9/02; G01N 27/3276; G01N 33/5308; G01N 33/5438

(57) ABSTRACT

A biomarker detection sensor includes a substrate; a working electrode formed by laser-scribing directly into the substrate so that a material of the substrate is transformed into graphene; a metal nanostructure formed on a graphene surface of the working electrode, wherein the metal nanostructure is shaped as a tree with plural branches extending away from the graphene surface; an aptamer covering a first surface area of the metal nanostructure; a reference electrode; and a counter electrode.

### CROSS-REFERENCE TO RELATED APPLICATIONS

This application is a divisional of U.S. patent application Ser. No. 17/924,418, filed on Nov. 10, 2022, which is a U.S. National Stage Application of International Application No. PCT/IB2021/054124, filed on May 13, 2021, which claims priority to U.S. Provisional Patent Application No. 63/024,756, filed on May 14, 2020, entitled “ELECTRODEPOSITED METAL MODIFIED LASER SCRIBED GRAPHENE ELECTRODES,” the disclosures of which are incorporated herein by reference in their entirety.

### BACKGROUND

### Technical Field

Embodiments of the subject matter disclosed herein generally relate to laser-scribed graphene (LSG)-based electrodes for sensing, and more particularly, to a metal nanostructured modified LSG-based electrode that can be used as a sensing platform for disease biomarker detection.

### Discussion of the Background

The modern diagnostics technology revolution called “precision health” has already witnessed a widespread deployment and growing interest in point-of-care (POC) and wearable non-and minimally-invasive diagnostic devices. Early, low-cost, easy-to-use, and accurate POC detection of disease biomarkers is critical for managing global health issues. The main reason for the increase in POC devices'demand is the preference for rapid diagnosis at outpatient healthcare, hospitals, critical care centers, and home healthcare. Therefore, there is a high demand to develop new and improved diagnostic tools to meet end-users'growing needs and the continuously evolving POC market.

Electrochemical biosensors have great potential to meet this demand due to their high specificity, accuracy, ease of use, and integration into handheld devices or mobile phone technology and the internet of things (IoT). For the development of electrochemical biosensors to meet the POC market's increasing demand, new electrodes that can be used to fabricate various sensing systems with improved electrochemical performance are highly desirable.

Laser scribing of different substrates, such as polymers or graphene oxide, emerged as a new method for mask-free and straightforward 3D patterned graphene production [1, 2]. The production of LSG or laser-induced graphene (LIG) electrodes using laser scribing of polyimide (PI) has been proved as a highly effective method of LSG production because of the low level of defects and large electrochemically active surface area due to the formation of stacked graphene flakes [3-5]. The LSG fabrication method creates flexible electrodes with fast and scalable fabrication without any complicated steps using mask or lithography equipment. A recent report showed that LSG electrodes provide many advantages such as mask-free synthesis, 3D porous morphology, large surface area, fast electron mobility, cost-effectiveness, and high electrocatalytic activity as compared to commercially available screen-printed carbon electrodes, glassy carbon electrodes, and carbon paste electrodes.

Various enzymatic or non-enzymatic LSG-based sensors have been reported for the detection of different disease biomarkers, neurotransmitters, ions, bacteria, and other biomolecules. Fenzl et al. (Fenzl, C., Nayak, P., Hirsch, T., Wolfbeis, O. S., Alshareef, H. N., Baeumner, A. J., 2017, Laser-scribed graphene electrodes for aptamer-based biosensing. ACS Sens. 2 (5), 616-620) reported a label-free voltammetric biosensor to detect the coagulation factor thrombin using LSG as a working electrode, an Ag/AgCl reference electrode, and platinum wire as a counter electrode. Although these studies demonstrated the LSG electrodes'potential for biosensor applications, for LSG electrodes to be qualified as POC electrochemical biosensor devices, a three-electrode system should be integrated on the same chip, and efforts in this direction will pave the way for the development of LSG-based POC devices.

In this context, LIG or LSG electrodes were modified with gold, polyaniline (PANI), reduced graphene oxide (rGO), and PANI or rGO further modified with gold [6]. In all cases, gold electroplating or polyaniline coating was carried out on interdigitated LSG electrodes using external platinum electrodes. Electrochemical impedance was used in combination with principal component analysis (PCA) to develop a multi-flavor detection sensor [6]. In another study, [7] developed laser-induced spherical noble metal nanoparticles (Au, Ag, and Pt) modified LIG electrodes using metal precursor-chitosan hydrogel ink coated on a PIsubstrate. These studies show a versatile method of producing different types of discrete and spherical noble metal nanoparticle modified LIG electrodes and used electrochemical impedance spectroscopy (EIS) to detect pathogens.

However, the existing sensors and methods are expensive, time-consuming and require highly skilled people to fabricate them. Thus, there is a need for a new sensor that can detect such biomarkers at an incredibly low price.

### BRIEF SUMMARY OF THE INVENTION

According to an embodiment, there is a biomarker detection sensor that includes a substrate, a working electrode formed by laser-scribing directly into the substrate so that a material of the substrate is transformed into graphene, a metal nanostructure formed on a graphene surface of the working electrode, wherein the metal nanostructure is shaped as a tree with plural branches extending away from the graphene surface, an aptamer covering a first surface area of the metal nanostructure, a reference electrode, and a counter electrode.

According to another embodiment, there is a biomarker detection sensor that includes a substrate, a working electrode formed by laser-scribing directly into the substrate so that a material of the substrate is transformed into graphene, a metal nanostructure formed on a graphene surface of the working electrode, wherein the metal nanostructure is shaped as a tree with plural branches extending away from the graphene surface, a polymer covering the graphene surface of the metal nanostructure, wherein plural cavities are formed in the polymer with a biomarker to be detected, a reference electrode, and a counter electrode.

According to still another embodiment, there is a method for making a biomarker detection sensor. The method includes providing a polyimide substrate, scribing with a laser beam into the polyimide substrate to form a graphene working electrode, depositing a metal by electrochemical deposition on the graphene working electrode to form a metal nanostructure that extends as a tree with branches from a surface of the graphene working electrode, adding a biomarker and a polymer to the surface of the graphene working electrode, and removing the biomarker to form corresponding cavities into the deposited polymer.

According to yet another embodiment, there is a system for determining a biomarker, and the system includes a biomarker detection sensor, a signal analyzer configured to directly connect to the biomarker detection sensor to receive measurements and generate a signal indicative of the biomarker, and a portable computing device that receives the signal and displays the signal on a screen. The biomarker detection sensor includes a working electrode formed by laser-scribing directly into a substrate so that a material of the substrate is transformed into graphene, a metal nanostructure formed on a graphene surface of the working electrode, wherein the metal nanostructure is shaped as a tree with plural branches extending away from the graphene surface, and a polymer covering the graphene surface of the metal nanostructure, wherein plural cavities are formed in the polymer with a biomarker to be detected.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of a laser-scribed graphene based electrochemical sensing electrode;

FIGS. 2A to 2D show the manufacturing steps of the laser-scribed graphene based electrochemical sensing electrode;

FIGS. 3A and 3B show a laser-scribed graphene electrode;

FIGS. 4A to 4E illustrate a laser-scribed graphene sensor that uses a metal nanostructure with an aptamer for detecting a biomarker;

FIGS. 4F to 4I illustrate a laser-scribed graphene sensor that uses a metal nanostructure with a polymer for detecting a biomarker;

FIGS. 5A to 5D illustrate scanning electron microscope (SEM) images of the working electrode of various biomarker detection sensors;

FIG. 6A shows the XRD spectra of an LSG electrode, LSG with gold nanostructure, LSG-AuNS, LSG with gold nanostructure and a polymer layer, LSG-AuNS-NIP, and LSG with gold nanostructure and a molecularly imprinted polymer, LSG-AuNS-MIP;

FIG. 6B shows the cyclic voltammograms obtained for LSG, LSG-AuNS, LSG-AuNS-MIP adduct electrode, and LSG-AuNS-MIP electrode;

FIG. 6C shows Nyquist plots obtained for the LSG, LSG-AuNS, LSG-AuNS-MIP adduct, LSG-AuNS-MIP electrodes after removal of the human epidermal growth factor receptor 2 (Her-2), and LSG-AuNS-MIP after binding 10 ng/ml of Her-2;

FIG. 7 shows the cyclic voltammograms obtained for LSG and LSG-AuNS electrodes prepared using electrochemical deposition of HAuCl₄ solution at different applied voltages and measured in 2.5 mM [Fe(CN)₆]^{3-/4-} containing 0.1 M KCl;

FIG. 8 is a histogram showing the effect of applied voltage on electrochemical response of the LSG-AuNS electrode;

FIG. 9 shows the electrochemical response obtained for the LSG-AuNS electrode prepared using different concentrations of HAuCl₄;

FIG. 10 shows the effect of electrodeposition time on the electrochemical response of the LSG-AuNS electrode;

FIG. 11A shows histograms of the current difference between LSG-AuNS-MIP adduct and LSG-AuNS-MIP after extraction of different Her-2 concentrations;

FIG. 11B shows histograms of the difference in oxidation current intensity between LSG-AuNS-MIP adduct and LSG-AuNS-MIP after binding Her-2 for different removal agents used;

FIG. 12A shows square-wave voltammograms of LSG-AuNS aptasensor for different concentrations of Her-2 measured in 2.5 mM [Fe(CN)₆]^{3-/4-} containing 0.1 M KCl;

FIG. 12B shows a corresponding curve obtained from different Her-2 concentrations (0.1 ng/ml-200 ng/ml);

FIG. 12C shows square-wave voltammograms obtained at LSG-AuNS-MIP incubated at different concentrations of Her-2;

FIG. 12D shows a corresponding calibration plot obtained for Her-2 concentration range of 1-200 ng/ml;

FIG. 13 shows analytical performances of the developed LSG-AuNS-MIP sensor compared to previously reported sensing systems;

FIG. 14A shows the response of the LSG-AuNS aptasensor in the presence of Her-2, cardiac Troponin-I (cTn-I), Cholesterol (Chol), Dopamine (DA), and Glucose (Glu);

FIG. 14B shows the electrochemical response of the LSG-AuNS-MIP sensor in the presence of Her-2, cTn-I, Chol, Gluc, and DA;

FIG. 15A shows the determination of Her-2 in various human serum samples when using the LSG-AuNS aptasensor;

FIG. 15B shows the determination of Her-2 in various human serum samples when using the LSG-AuNS-MIP sensor;

FIGS. 16A and 16B show a biomaker detection system that uses an LSG-AuNS based sensor;

FIG. 17 illustrates a response provided by the biomaker detection system of FIGS. 16A and 16B; and

FIG. 18 is a flow chart of a method for forming one of the sensors discussed above.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a gold nanostructured modified LSG electrode for determining a cancer related biomarker. However, the embodiments to be discussed next are not limited to a gold-based electrode, or to a sensor that determines only biomarkers, but may be applied to other metals and/or biological substances or materials.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

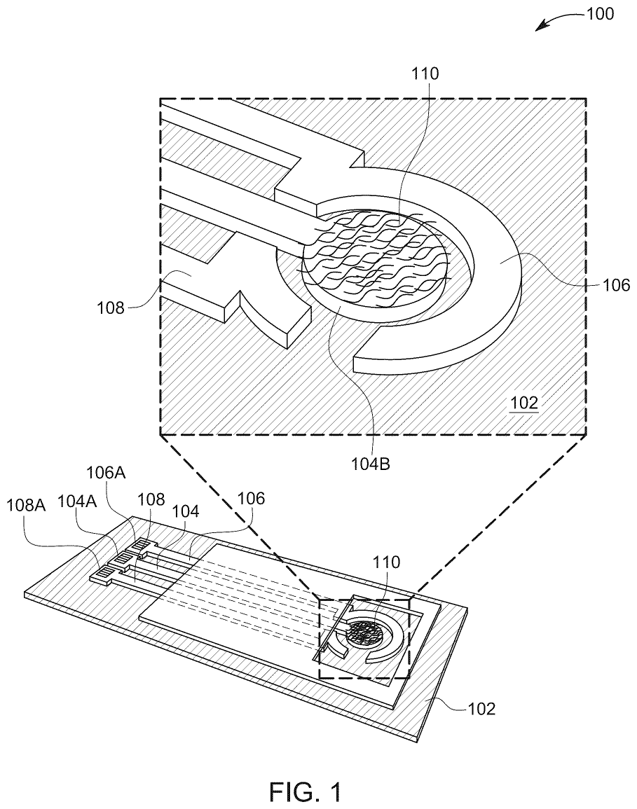

According to an embodiment, as illustrated in FIG. 1, a new class of nanostructured gold modified LSG (LSG-AuNS) electrochemical sensing electrode 100 is introduced and the electrode 100 includes an LSG-AuNS working electrode 104, an LSG counter electrode 106, and an LSG reference electrode 108, all of them located on a same substrate 102. Each electrode has a corresponding pad 104A, 106A, and 108B, which is electrically connected to an external device, for example, smart device, computer, processor, etc. The LSG-AuNS electrode 104 is processed by electrodeposition of gold chloride (HAuCl₄) solution, which generates Au nanostructures 110, which results in a 2-fold enhancement in sensitivity and electrocatalytic activity compared to bare LSG electrode and commercially available screen-printed gold electrode (SPAuE). Note that other metals than Au may be used for electrodeposition. For example, in one embodiment, Palladium is electrodeposited on the electrode 104. The LSG-AuNS electrochemical aptasensor shows good qualities for detecting Her-2 with a limit of detection (LOD) of 0.008 ng/ml and a linear range of 0.1-200 ng/mL. The LSG-AuNS aptasensor can easily detect different concentrations of Her-2 in undiluted human serum. In one application, the LSG-AuNS sensor system's potential to develop POC biosensor devices is exemplified by integrating the LSG-AuNS electrodes with a handheld electrochemical system operated using a custom-developed mobile application. These features are now discussed in more detail.

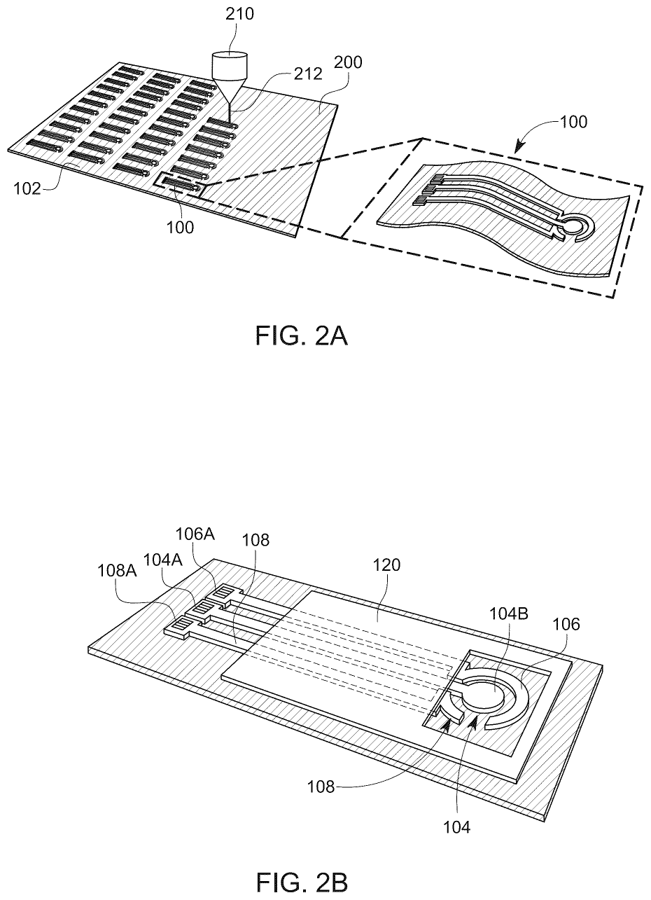

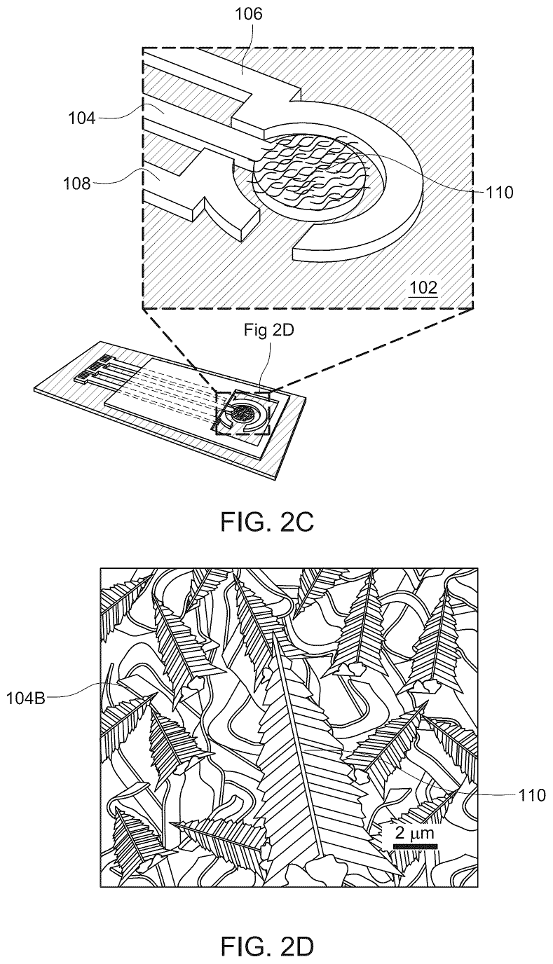

The gold nanostructured modified LSG based electrochemical sensor 100 was obtained as illustrated in FIGS. 2A to 2C. More specifically, a sheet of PI 200 was used as a starting point as shown in FIG. 2A. A laser 210 was used to generate a laser beam 212, which impinges on the top surface of the PI sheet 200, thus changing its properties and forming the LSG electrodes 104 to 108 of the sensor 100, as also shown in FIG. 2A. Note that the top surface 104B of the tip of the working electrode 104 is configured to have a circular perimeter. An insulator coating 120 was applied on top of part of the electrodes 104 to 108, as shown in FIG. 2B, so that the pads 104A to 108A are left uncovered and also the tips of the electrodes. An electrodeposition technique was then applied to the configuration shown in FIG. 2B to generate 3D gold nanostructures 110 on the porous LSG electrode 104's surface 104B and obtained an excellent surface coverage of gold. In one application, the entire round tip surface 104B of the working electrode 104 is covered with the 3D gold nanostructures 110, as shown in FIG. 2C.

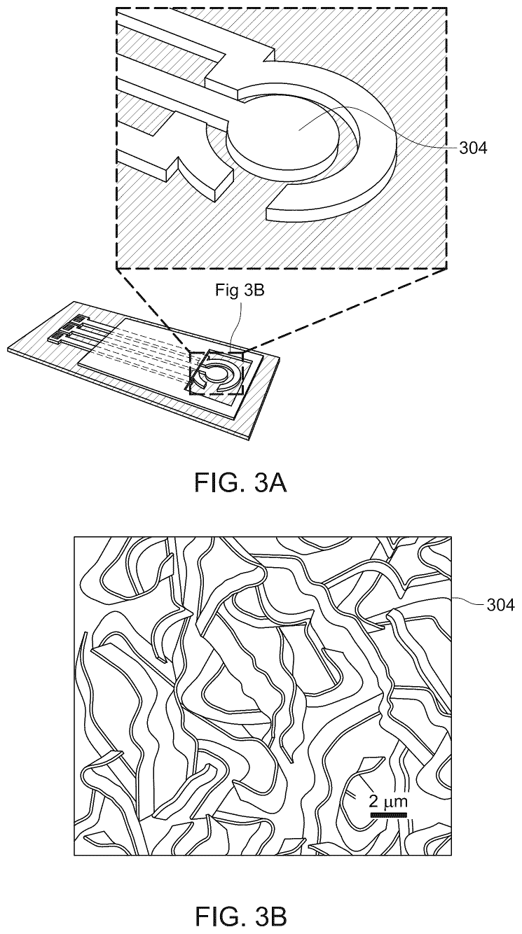

The method employed to generate these nanostructures 110 produces a unique morphology of spiky and Christmas tree-like 3D shaped gold nanostructures 110, which is present on the surface 104B of the working electrode 104 with better surface coverage, which are readily available for immobilization of biorecognition molecules. In this regard, FIG. 2D shows the spike and Christmas tree-like 3D shaped gold nanostructure 110 extending away from the surface 104B of the working electrode 104. This structure is very different from the gold nanoparticles deposited by [7] on the working electrode as the extended structure 110, which is separated and away from the surface 104B of the working electrode 104, is more exposed to interact with biological material. Due to gold's 3D nanostructures 110, the electron transfer rate is higher in the LSG-AuNS electrode 104 than the simple LSG electrode 304 illustrated in FIG. 3A, which does not have the gold nanostructures 110. The same is true for an electrode that has only spherical gold particles distributed over its surface.

It is noted that FIG. 3B, which shows in more detail the structure of the electrode 304, without the nanostructure 110, has no spiky or Christmas-tree like formations. These unique features of the sensor 100 enable to develop a low-cost, highly sensitive aptasensor with a good reproducibility. The electro-chemical biosensing system 100 discussed herein does not require printing an external Ag/AgCI reference electrode and platinum counter electrode for electrodeposition and biosensor applications.

For comparing the novel sensor 100 with existing sensors, in one embodiment, the LSG sensor 100 was designed and fabricated as a three-electrode system with dimensions as follows: length of 2.8 cm, width of 1.2 cm, and radius of 1.5 mm. The electrodes 104 to 108 were formed by CO₂ laser writing on a pre-cleaned PI sheet. All three LSG electrodes (working, reference, and counter) were fabricated on the same PI substrate 102. The scribing process was performed under inert gas flow to minimize the heteroatom binding to the graphene surface. In this embodiment, the optimal laser scribing parameters used were 3.2 W power, 2.8 cm/s speed, 1000 pulses per inch, and 2.5 mm Z distance to obtain a relatively low sheet resistance value (58 Ω/square).

The bare LSG electrode 104 was modified by electrochemical deposition using chronoamperometry, applying a constant potential of −0.9 V for 240 s in a solution containing 50 mM HAuCl₄ prepared in 0.5 M HCl as the electrolyte.

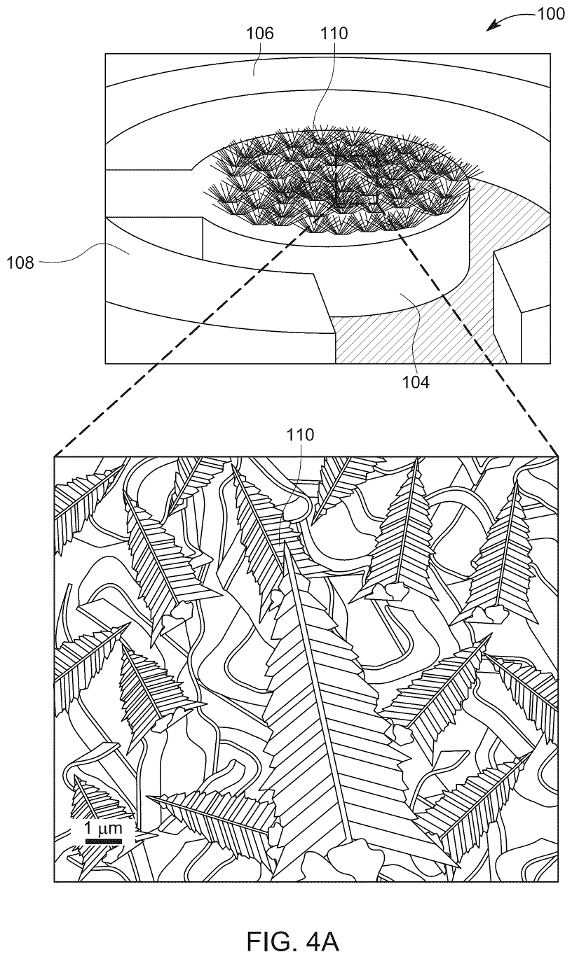

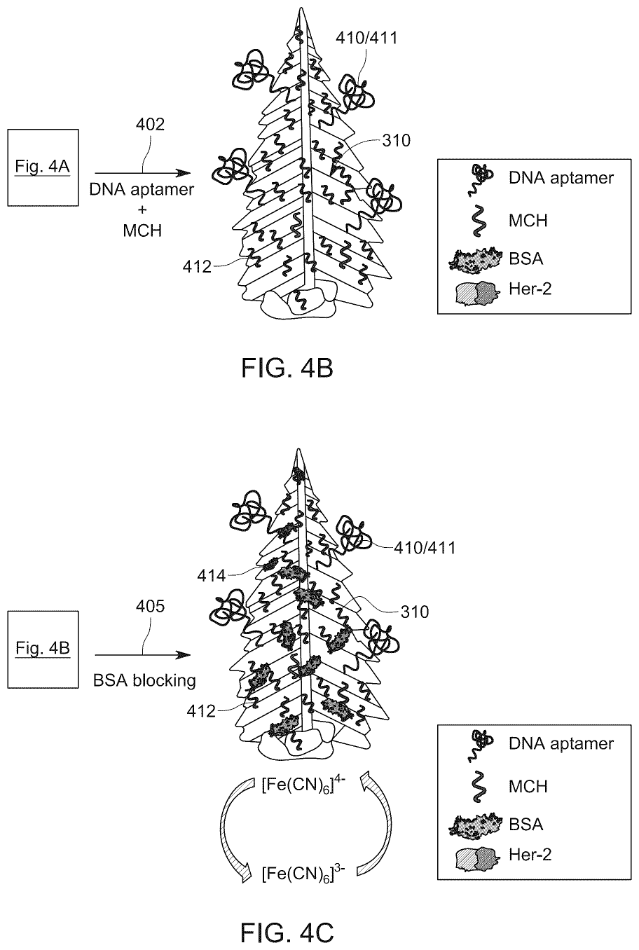

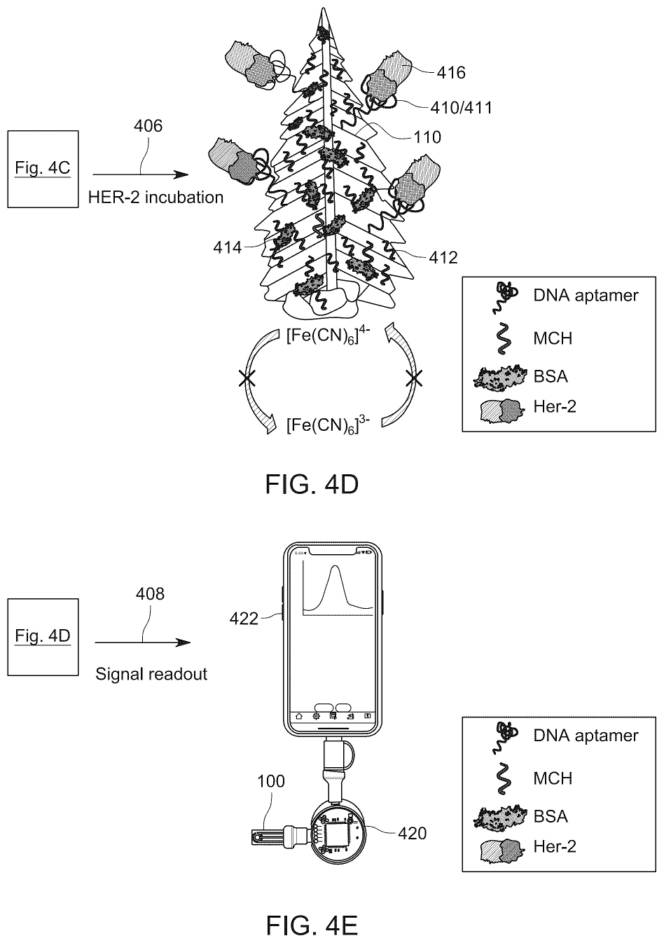

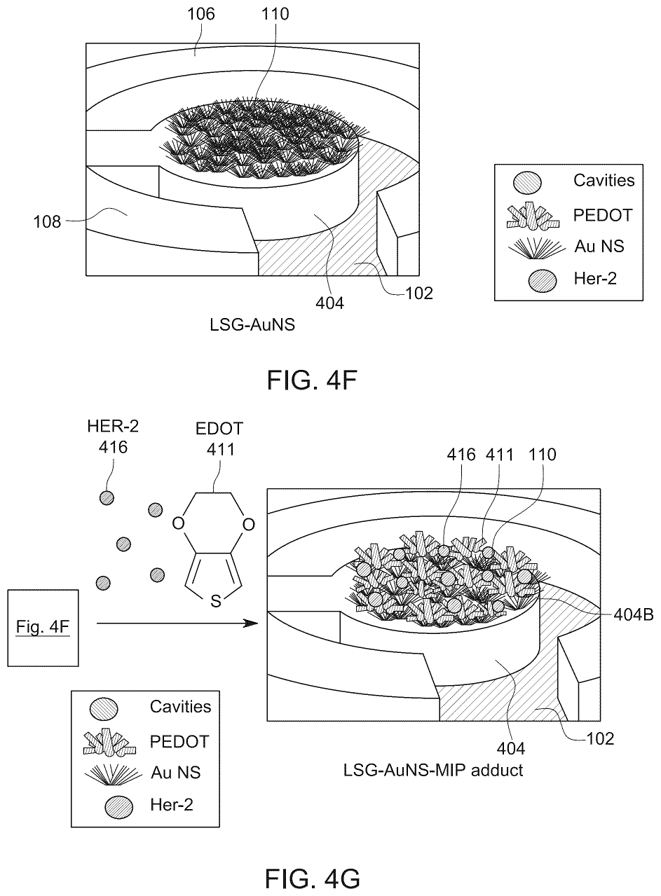

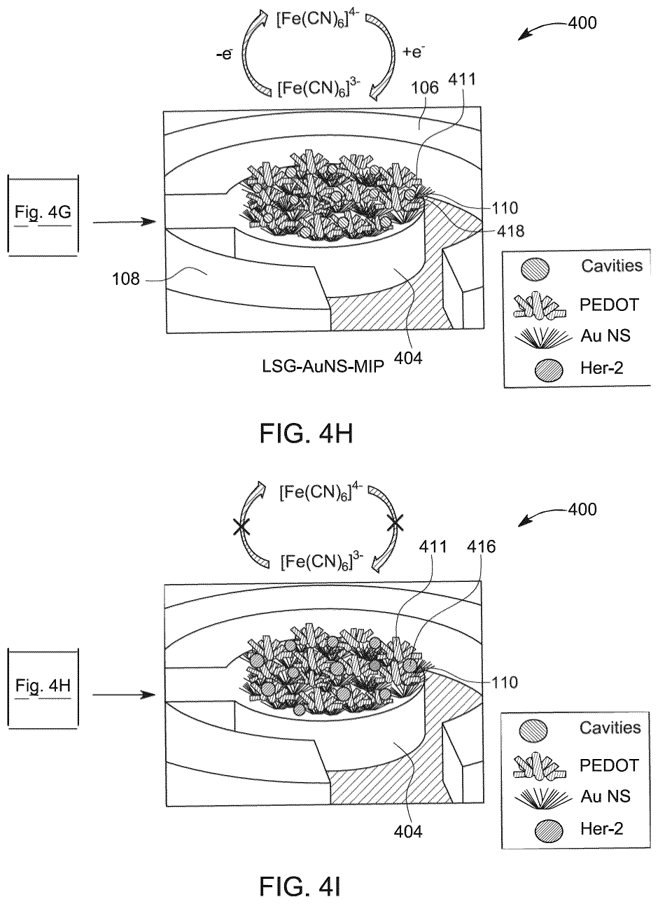

Finally, the 3D AuNS modified LSG 104 was rinsed with ultrapure water and dried with nitrogen gas to obtain the nanostructures 110, as shown in FIG. 4A.

Stock solutions of DNA aptamer (100 μM) 410 were prepared in a TE buffer (10 mM TRIS, pH 8) and stored at −20° C. An aptamer is defined herein as oligonucleotide or peptide molecules that bind to a specific target molecule. In this embodiment, thiol modified Anti-Her-2 DNA aptamer (APT) has the structure [ThiC6]AACCGCC-CAAATCCCTAAGAGTCTGCACTTGTCATTTTGTA-TATGTATTTGGTTTTTGGCTCTCACAGACACACTACACACGCACA. The Fresh working solutions of DNA aptamer 410 were prepared using 10 mM PBS at pH 7.4 and were used immediately. The concentrated Mercaptohexanol (MCH) solution was first prepared in ultrapure water and then further diluted in 10 mM PBS (pH 7.4). 4 μM of DNA aptamer 410 was mixed with 20 μM of MCH 412 prepared with 10 mM PBS (pH 7.4) to obtain a homogenized solution. As the next step, 6 μL from this mixture was placed in step 402 onto the LSG-AuNS modified working electrode 104 and incubated for 16 h, as shown in FIG. 4B. The MCH 412 covers a first part of the surface of the nanostructure 110 while the aptamer 410 covers a second part of the surface of the nanostructure 110. However, a third part of the surface of the nanostructure 110 is not covered by either of the MCH or the aptamer. The LSG-AuNS electrode surface was cleaned with 10 mM PBS (pH 7.4) to remove the excess of aptamer molecules 410 and MCH 412. The aptasensor was incubated in step 405 in 0.1 mg/mL of BSA solution 414 in 10 mM PBS (pH 7.4) for 45 min to cover a third part of the surface of the nanostructure 110, to reduce non-specific adsorption, as illustrated in FIG. 4C. In one application, the sum of the surfaces of the first to third parts covers the entire surface of the nanostructure 110. However, in another application, the sum of the first to third parts cover less than the entire surface of the nanostructure 110. Finally, the aptamer modified electrode 104 was washed with 10 mM PBS (pH 7.4) for at least five times. After the aptamer immobilization, 0.1, 1, 10, 50, 100, and 200 ng/ml of Her-2 416 were prepared in 10 mM PBS (pH 7.4) as the analyte and placed on top of the LSG-AuNS/aptamer electrode 104 in step 406 for 1 h incubation, as shown in FIG. 4D. The electrode was then rinsed with PBS to remove non-bonded Her-2 and was connected to a electrochemical analyzer 420 in step 408, for measurements, as shown in FIG. 4E. A smart phone 422 with a corresponding application were used for processing the measurements. For the detection of Her-2 in undiluted serum, different concentrations of Her-2 were placed into the serum and incubated onto the electrode 104's surface for 1 h, and washed with 10 mM PBS (pH 7.4) before electrochemical measurements.

In another embodiment, the aptamer 410 can be replaced with a molecularly imprinted polymer (MIP) to detect the Her-2 protein. MIPs have many advantages such as low-cost, high stability, selectivity, and robustness. Thus, all of these factors make the MIPs very promising alternatives to build highly selective sensors. In this context, electropolymerization has found application in the synthesis of MIPs for proteins in aqueous solutions providing several advantages such as the control of the thickness of the polymer film and reducing the MIP synthesis time. In recent years, MIP based electrochemical sensors demonstrated their ability as a promising analytical tool for the detection of cancer biomarkers [8].

In this embodiment, the 3,4-ethylenedioxythiophone (EDOT) was used as the polymer 411. Other polymers may be used for the MIP, for example poly-EDOT (PEDOT). The modification of the LSG-AuNS 104 using the MIP 411 was achieved through several steps, as illustrated by FIGS. 4F to 4K. In this respect, note that the aptamer 410 in FIGS. 4A to 4E is replaced by the polymer 411 in FIGS. 4F to 4K. First, the desired analyte was incubated for 15 min on the surface 404B of the working electrode 404 as show in FIG. 4G. A 5 μL of a solution with 0.4 mg/mL Her-2 416 was found to be sufficient to create enough MIP cavities 418 (see FIG. 4H) within the polymer network 411. After the protein's adsorption step, about 70 μL of 10 mM EDOT 411 was gently placed on the sensing zone 404B covering all exposed areas of the electrode 404, as shown in FIG. 4G. The EDOT electropolymerization is achieved by the chronoamperometry technique at +0.85 V for 70 s. After that, the working electrode 404 was washed gently with DI water and air-dried for 5 min. Upon complete dryness, the template molecule 416 was extracted from the polymer network 411 using ethanol on the top of the working LSG-AuNS-MIP adduct electrode 404 for 20 min to form MIP cavities 418, as shown in FIG. 4H. Note that the cavities 418 correspond to the extracted molecule 416, and thus, the shape of the cavities 418 matches the shape of molecules 416. The formed sensor 400 was then exposed to Her-2 for rebinding of the Her-2 416 onto the MIP sensor, as shown in FIG. 4I. Due to the selective capture and binding of Her-2 416 onto the MIP cavities 418, the diffusion of the 2.5 mM ferri/ferrocyanide redox probe in 0.1 M KCl to the electroactive area of the MIP sensor 400 was hindered, leading to the decrease in current intensity of the electrical signal. This step also shows the possible integration of the LSG-AuNS-MIP sensor 400 into a homemade POC device.

Various qualities and features of the LSG-AuNS electrode 104 and the LSG-AuNS-MIP electrode 404, and the corresponding sensors 100 and 400 are now discussed. The electrochemically active surface area 104B of the novel LSG-AuNS electrode 104 was compared with the commercially available screen-printed gold electrodes (SPAuE) mentioned in [13]. The obtained results indicated that LSG-AuNS electrodes have a high electroactive surface area due to the 3D gold nanostructures and thus have promising potential as a platform for biosensing applications. More specifically, a cyclic voltammetry (CV) method with a scan rate of 100 mv/s and sweeping the potential from −0.6 V to +0.4 V and square wave voltammetry (SWV) method with a frequency of 2 Hz and sweeping the potential from −0.5 V to +0.5 V were used to study electrochemical responses of the LSG-AuNS/aptamer electrodes 104 before and after interaction with the Her-2. The EIS parameters used in this embodiment were a frequency from 1.0 Hz to 100 kHz at 0 V. All electrochemical measurements were performed at room temperature in 0.1 M KCl containing 2.5 mM [Fe(CN)₆]³⁻/[Fe(CN)₆]⁴⁻ as a redox probe with LSG reference and counter electrodes. The changes in the current intensities are correlated to the different amounts of Her-2 captured by the DNA aptamer immobilized on the electrode surface. All electrochemical measurements were performed in triplicate.

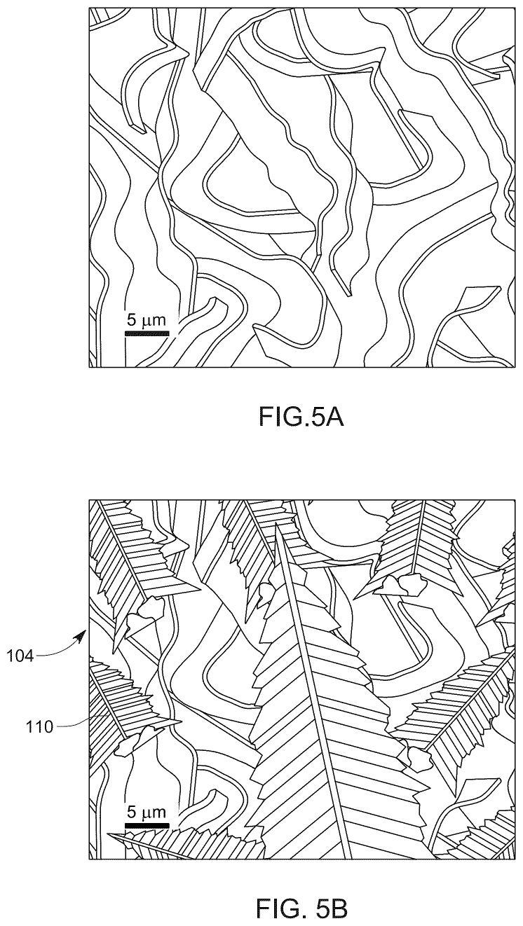

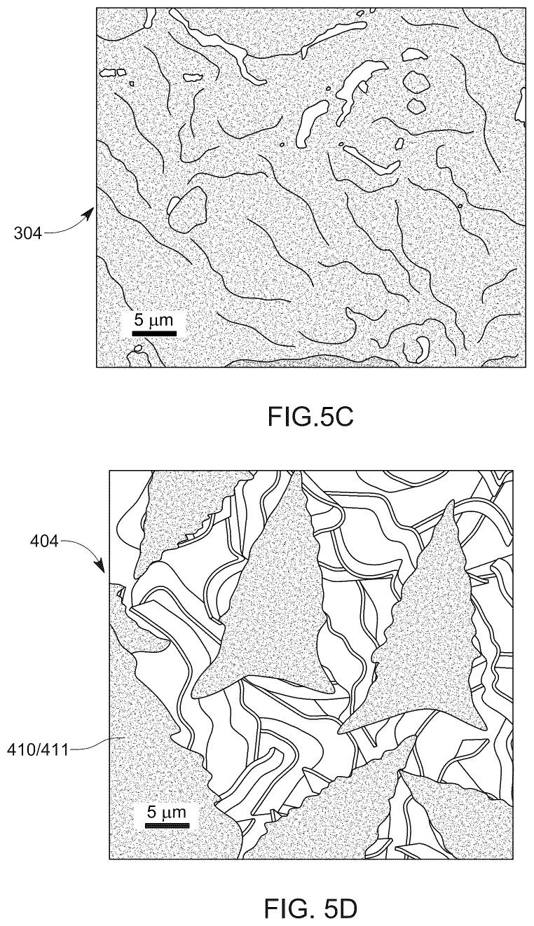

FIGS. 5A to 5D show the SEM images of the traditional LSG electrode, the LSG-AuNS electrode 104, the AuSPE electrode 304 (shown in FIGS. 3A and 3B and corresponding to [13]), and the LSG-AuNS-MIP electrode 404. It can be seen in FIG. 5A that the highly porous 3D structure of the LSG electrode was formed by the irradiation of the PI substrates, which is in agreement with the previous studies. After the electrochemical deposition of gold on the LSG working electrode surface, it was observed full surface coverage of the LSG-AuNS working electrode 104 by the gold nanostructures, as shown in FIG. 5B. Spiky and Christmas tree-like shapes 110 were observed for the AuNS deposited onto the LSG compared to the commercially available SPAuE, which is shown in FIG. 5C. Note that not a single spiky and Christmas tree-like shape 110 was observed on the AuSPE electrode 304 of [13]. FIG. 5D shows that all the spiky and Christmas tree-like shapes 110 of the LSG-AuNS-MIP electrode 404 were covered by the polymer 411.

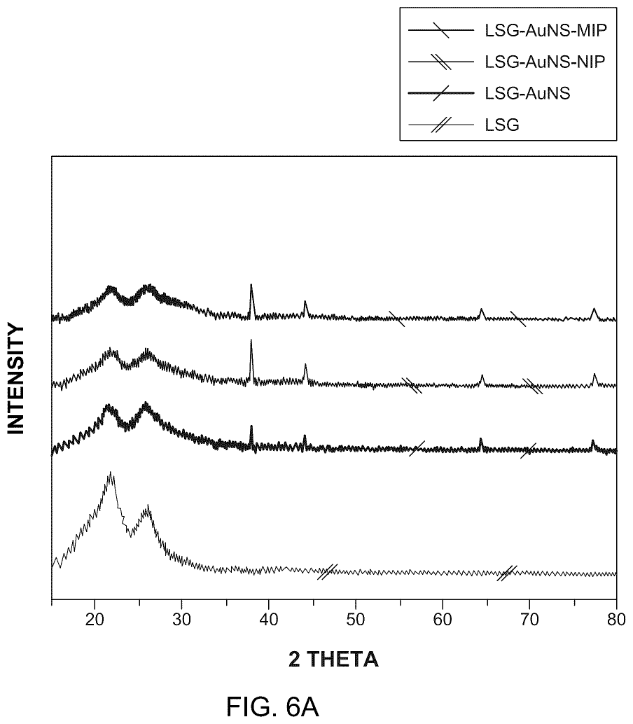

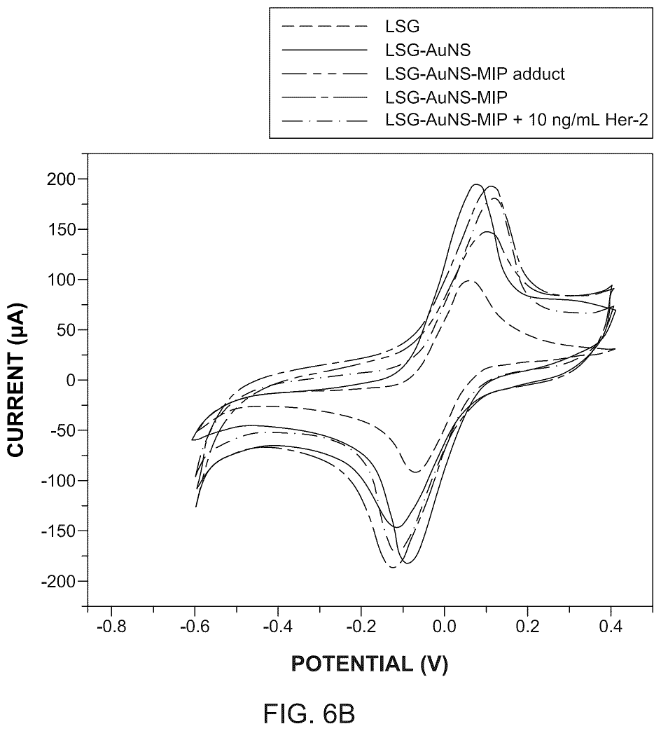

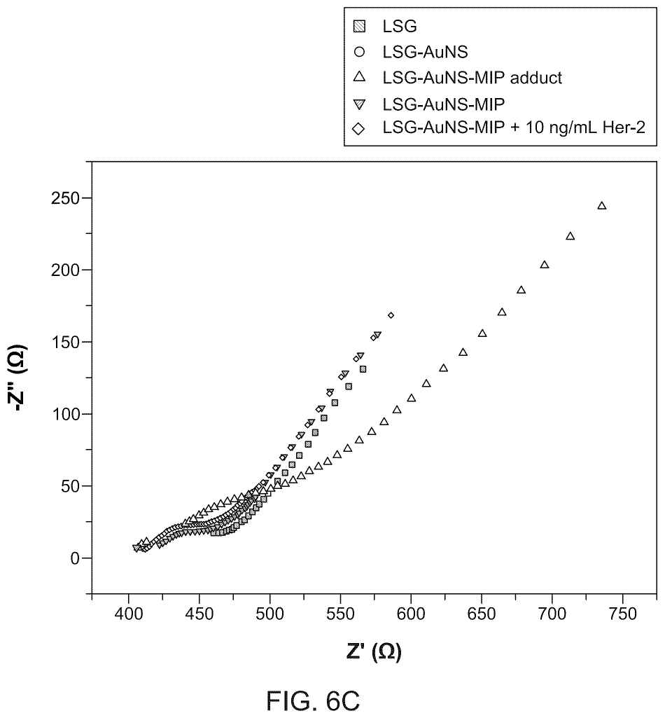

FIG. 6A presents the x-ray diffraction (XRD) spectra of the LSG, LSG-AuNS, LSG-AuNS non-imprinted polymer (NIP), and LSG-Au-MIP electrodes. The LSG-AuNS-NIP is obtained by electropolymerization with EDOT or PEDOT of the LSG-AuNS electrode 404 followed by no adsorption and extraction of the Her-2 (see FIGS. 4G and 4H) and thus no MIP cavities 418 for the LSG-AuNS-NIP electrode. The crystallinity of the LSG and LSG-AuNS before and after electrochemical deposition of EDOT was characterized using the XRD technique.

The XRD results showed two prominent peaks in the 2θ region of 21.9° and 25.5° attributed to a (002) plane with a high graphitization degree. The XRD spectrum of LSG-AuNS exhibited a high degree of crystallinity of the gold nanostructures covering the LSG. All the peaks are matched well with the gold nanostructures in the 2θ region (38.31°(1 1 1), 44.46° (2 0 0), 64.67° (2 2 0) and 77.45° (3 1 1)) and found to be identical with those reported for the standard gold metal according to JCPDS database. Therefore, these results suggest that the gold nanostructures 110 are crystalline. After modification of the LSG-AuNS surface with the PEDOT (NIP) and the MIP film, similar peaks are found, indicating that the PEDOT does not affect the crystallinity of the LSG-AuNS.

The electrochemical characterization of the developed LSG-Au-MIP sensor 400 was performed using CV and electrochemical impedance spectroscopy (EIS) techniques as indicated in FIGS. 6B and 6C, respectively. The obtained results confirmed the successful formation of the MIP cavities 418 on the polymer network 411 capable of capturing the Her-2 target analyte. After the deposition of gold nanostructures 110 on top of the LSG working electrode 404, a significant enhancement of the current intensity was observed due to the high conductivity of the AuNS and their large surface area facilitating the transfer of electrons. In the next step, after the electropolymerization of the MIP adduct, a significant decrease in current intensity was observed due to the Her-2 protein 416 adsorbed on the LSG-AuNS and polymer matrix 411 (see FIG. 4G). Once the Her-2 416 was removed (see FIG. 4H), a significant increase in the current intensity was noticed due to the release of the imprinted cavities 418. After rebinding Her-2 with a 10 ng/ml of Her-2 (see FIG. 4K), a current intensity hindrance was noticed because of the occupation of the imprinted cavities 418 by the Her-2 target 416 and thus confirmed the selectivity of the polymer layer 411. Note that FIG. 6C shows Nyquist plots obtained for LSG, LSG-AuNS, LSG-AuNS-MIP adduct, LSG-AuNS-MIP after removal of Her-2, and LSG-AuNS-MIP after binding 10 ng/ml of Her-2. The EIS parameters used were frequency 1.0 Hz to 100 kHz at 0 V.

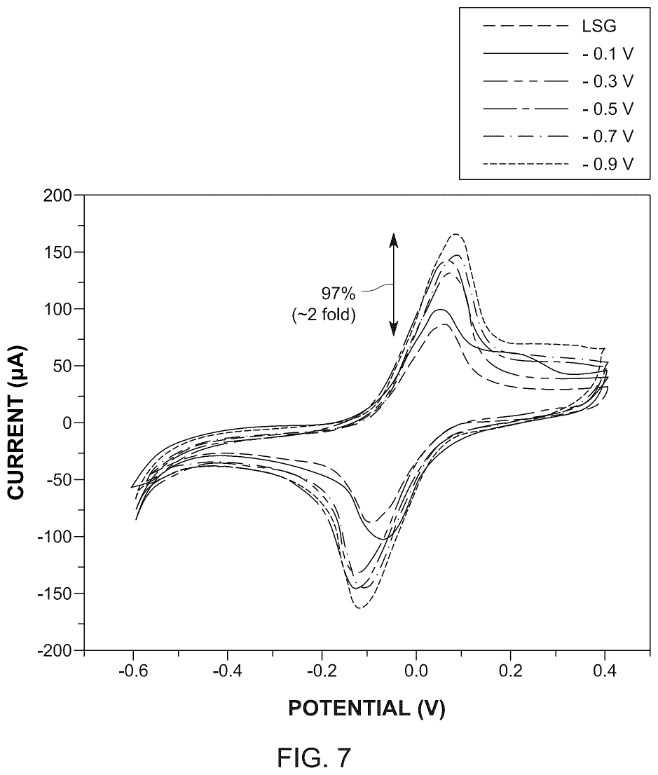

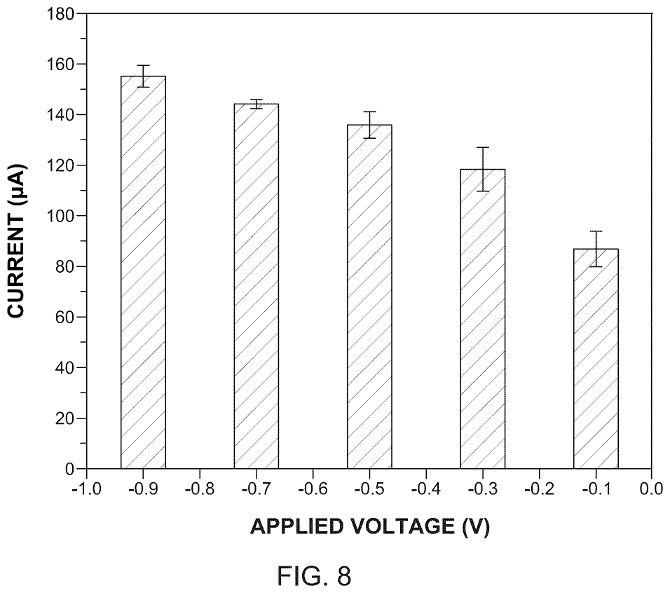

Although AuNS have been previously reported [9-12] onto different types of macro and microelectrodes and showed great potential for the fabrication of various types of biosensors due to the large surface area and ease of modification of the electrode surface with detection probes, these structures were never before deposited onto an LSG electrode so that the working electrode is fully covered with these structures. Considering the broader application of gold nanostructured electrodes, the inventors carried out the electrochemical deposition of gold onto the LSG working electrode for a sensor that also includes the reference and counter LSG electrodes, on the same chip. Several parameters were considered for the electrochemical deposition of the AuNS 110 over the entire surface of the working electrode 104, including the deposition time, the applied voltage, and the HAuCl₄ precursor concentration. The inventors found that the applied voltage for electrochemical deposition affects the surface coverage of the LSG-AuNS electrode 104 and its conductivity. Different electrochemical deposition potentials were tested in the range of −0.1 V to −0.9 V for 180 s in a 50 mM solution of HAuCl₄. The cyclic voltammograms presented in FIG. 7 demonstrate the highest current response for the LSG-AuNS electrode prepared using the potential of −0.9 V. Approximately a two-fold (97%) increase in the current response was observed after gold nanostructures deposition compared to the bare LSG electrode. As indicated in FIG. 8, the histogram shows that the increase in the applied potential (−0.1 V to −0.9 V) for the electro-chemical deposition of gold increases the electrochemical response of the LSG-AuNS sensor. The SEM characterization also showed an incomplete deposition of gold from −0.1 V to −0.7 V, and full surface coverage was obtained at −0.9 V.

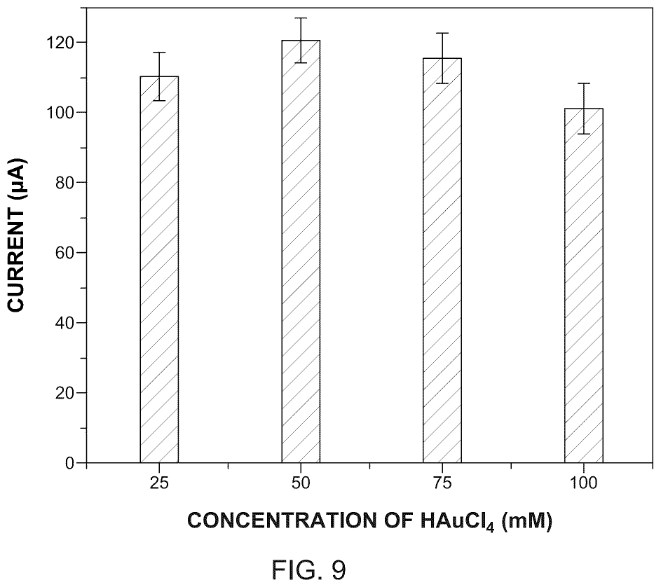

The HAuCl₄ concentration effect on the electrochemical performance of the LSG-AuNS electrode was also studied. Different HAuCl₄ concentrations were used, such as 25 mM, 50 mM, 75 mM, and 100 mM. As can be seen in FIG. 9, the results obtained showed the highest electrochemical response for the LSG-AuNS electrode 104 prepared using 50 mM of HAuCl₄. This is due to the full coverage of the LSG working electrode surface 104B by the AuNS 110, leading to a high electrocatalytic effect and large active surface area. However, after increasing the HAuCl₄ concentration beyond 50 mM, the electron transfer was hindered due to the agglomeration and formation of densely distributed AuNS 110. Notably, at higher concentrations, it was observed that some of the gold material was peeling off from the LSG-AuNS electrode. Hence, 50 mM of HAuCl₄ was chosen as the optimal value for the rest of the experiments.

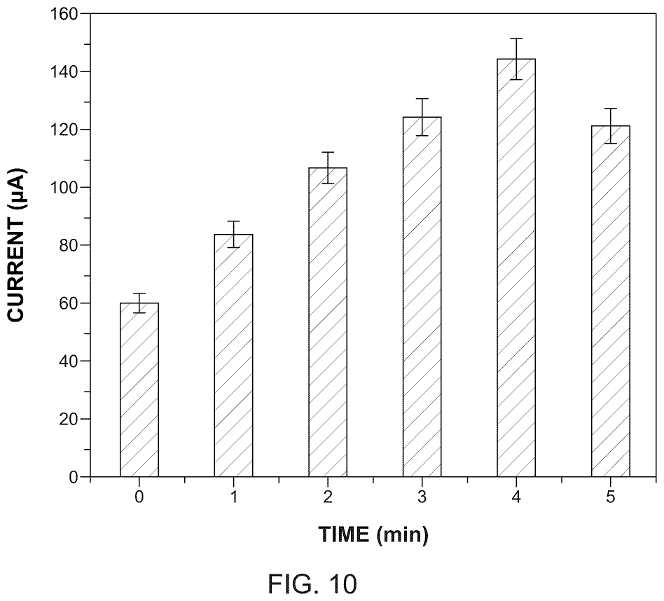

Another consequential parameter is the deposition time that affects the amount of the AuNS 110 deposited and surface coverage. To optimize the deposition time, the concentration of gold and the applied voltage were fixed as 50 mM HAuCl₄ and −0.9 V, respectively. The AuNS electrodeposition time was varied from 1 to 5 min to study its effect on the LSG-AuNS electrode performance. FIG. 10 shows the obtained results for different electrodes prepared using different electrodeposition times. It can be seen that 4 min deposition time yields the highest electrochemical response compared to other LSG-AuNS electrodes prepared using different gold deposition times. Therefore, 4 min was chosen as the optimal value for the electrodeposition time for the rest of the experiments.

The effect of the scan rate on the LSG and LSG-AuNS electrodes prepared under the optimal conditions found above was studied. The active surface area 104B of the LSG-AuNS electrode 104 was found to be 0.152 cm², which is greater than the LSG bare electrode, which is 0.086 cm², and also greater than the one commercially available SPAuE (0.078 cm²). The significant amplification of the active surface area is due to the high surface area of the AuNS 110 deposited on the LSG working electrode 104 and their excellent electrocatalytic effect. Thus, the LSG-AuNS electrode 104 could serve as a potential candidate for developing highly sensitive electrochemical sensors and biosensors.

The LSG, LSG-AuNS, and LSG-AuNS-MIP electrodes'flexibility was also investigated by bending these electrodes at different angles for 1 min. It was observed that the bending of the electrodes at about 45° and 90° did not affect their electrochemical responses. All the electrodes responses remained almost unchanged, proving the new electrode platform's flexibility.

For the system 400 that uses the PEDOT as a suitable polymer to prepare the polymer film 411 onto the LSG-AuNS working electrode 404, it is desired to optimize the PEDOT film deposition parameters. Indeed, several parameters such as the applied potential, the concentration of EDOT, and the electropolymerization time were optimized. In this embodiment, the parameters that yield the highest current response were chosen to fabricate the MIP sensor. The applied potential during the electropolymerization of EDOT was explored from 0.70 V to 0.90 V, where 0.85 V yielded the highest current intensity response. The monomer concentration (EDOT) was explored from 10 mM to 40 mM, where 10 mM was found to have the most negligible capacitance current, and thus it was chosen for further experiments. Finally, the polymerization time was explored between 70 s, 120 s, and 180 s where the former yielded the highest current intensity. Thus, the most optimal PEDOT electropolymerization parameters for the LSG-AuNS working electrode are 0.85 V in 10 mM EDOT for 70 s.

Once the PEDOT electropolymerization parameters were optimized, the next step is to optimize the imprinting process. Three most consequential parameters that affect the MIP film preparation on top of the LSG-AuNS electrodes were optimized in this embodiment. The first step is the adsorption step, where Her-2 is incorporated into the polymer matrix. The second step is the template extraction, where Her-2 is extracted from the polymer matrix to create the selective cavities that will be later used to capture the Her-2 target analyte. The third step is the rebinding, where Her-2 is reintroduced again to the sensor for detection.

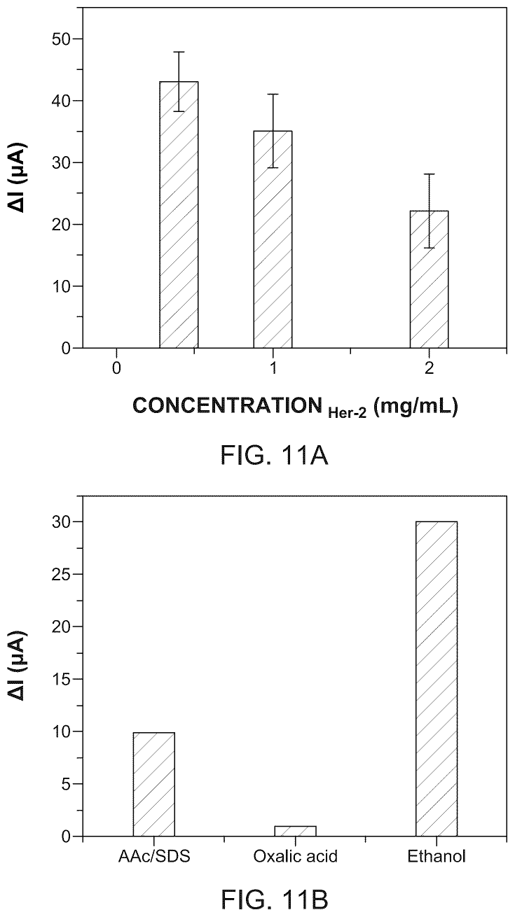

The concentration of the Her-2 protein adsorbed on the LSG-AuNS electrode 404 is the first parameter to be discussed now for optimization. A high concentration of the target analyte (Her-2) adsorbed on the LSG-AuNS might cause the aggregation leading to a decrease in the number of specific cavities and the sensitivity of the sensor. Hence, several concentrations of Her-2 have been tested to get the optimized sensor response. The adsorption time was explored between 20, 30, and 60 min with no observed significant differences. As such, the chosen time of incubation is 20 min for all subsequent data. FIG. 11A shows the histograms of the current difference between the LSG-AuNS-MIP adduct and LSG-AuNS-MIP after extraction for different electrodes prepared using different concentrations of Her-2 protein during the adsorption. It was found that 0.4 mg/mL allows the formation of imprinted cavities and easiness of removing Her-2 from the polymer matrix. Several strategies have been tested to remove Her-2 from the LSG-AuMIP adduct, including the use of acetic acid and SDS (0.5%) for 30 min, oxalic acid (0.5 M) for overnight, and pure ethanol for 20 min. The selection of the removal agent is of high interest, and it should extract the target analyte 416 without damaging the imprinted cavities 418. After using the three different strategies to remove Her-2 from MIP adduct, 10 ng/ml of Her-2 was incubated, and the measurements were performed using CV in a 0.1 M KCl containing 2.5 mM ferri/ferrocyanide solution as a redox probe. The results showed a difference in current intensity between the LSG-AuNS-MIP after removal of Her-2 and the LSG-AuNS-MIP after binding 10 ng/ml of Her-2 using the three different strategies of template removal, as illustrated in FIG. 11B. The obtained results showed that pure ethanol exhibited the best performance in terms of efficiency and less time of removal that leads to the creation of more cavities 418 in the polymer film 411.

The aptamer immobilization on the LSG-AuNS aptasensor 100 was tested in the presence and absence of the MCH 412. It was found that in the presence of the MCH, the electrochemical response of the aptasensor 100 was higher compared to the aptasensor response in the absence of MCH. The immobilization of the DNA aptamer 410 in the presence of the MCH 412 resulted in the proper folding and minimum steric hindrance of the DNA, which supports better attachment of the Her-2 protein 416 on the LSG-AuNS-DNA aptamer electrode surface 104B. The incubation time is another parameter that needs to be optimized for the best performance of the aptasensor. In particular, the inventors incubated the aptasensor 100 in 10 mM PBS containing 100 ng/ml of Her-2 by varying the incubation time from 15 to 60 min. The aptasensor showed a measurable response after 15 min incubation time, and there was no statistically significant variation observed from 15 min to 60 min incubation time.

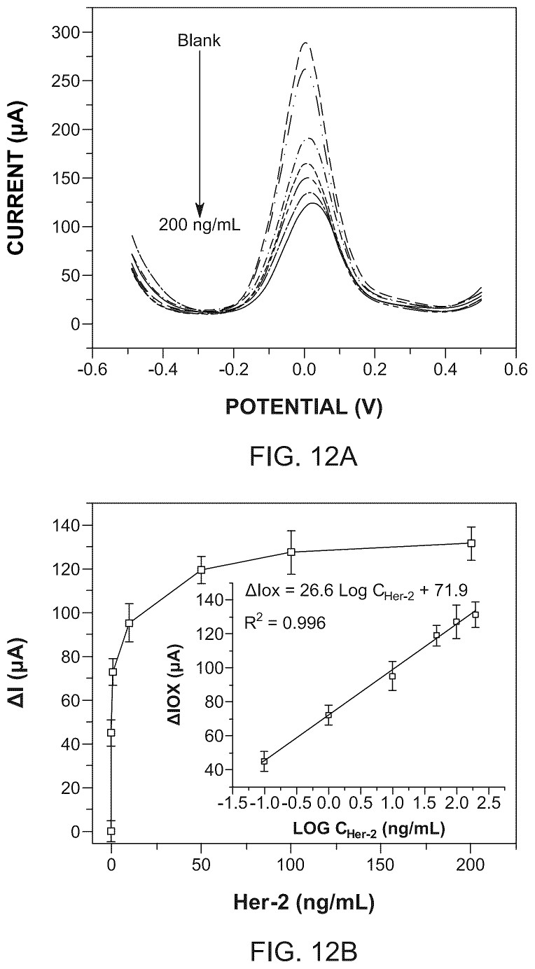

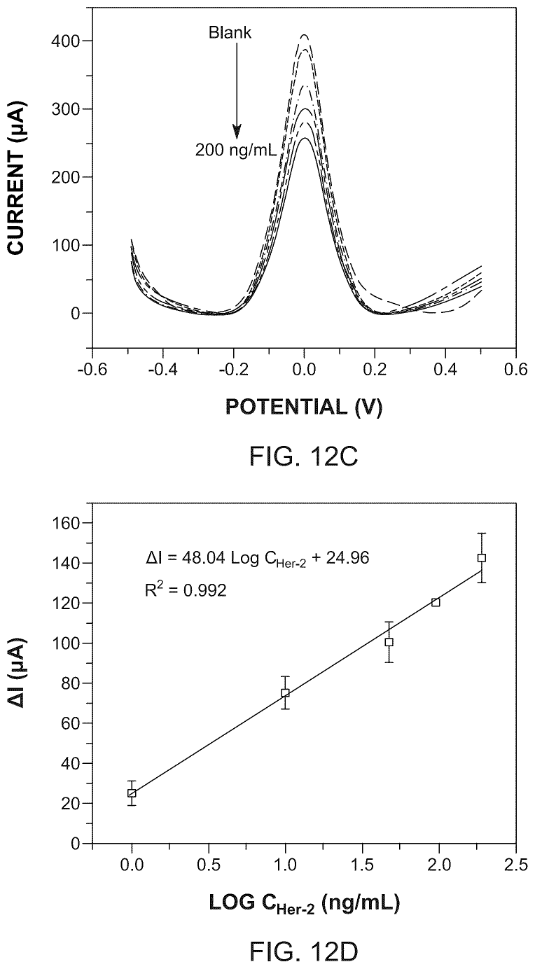

Under the above found optimized experimental conditions, including the AuNS 110 deposition, the immobilization of the aptamer, and the incubation time for Her-2 binding, the inventors investigated the sensitivity of the developed aptasensor 100 towards detecting the Her-2 protein. The proposed aptasensor 100 showed a decrease in the electrochemical response of [Fe(CN)₆]^{3-/4-} redox probe with the increase of the Her-2 concentration, as illustrated in FIG. 12A. The decrease in the response of the aptasensor with the increasing concentration of the Her-2 is due to the hindrance in the diffusion of the redox probe to the electrode surface, i.e., the higher the amount of Her-2 bind to the electrode surface increases the hindrance in the diffusion process. FIG. 12B presents the corresponding calibration plot obtained for the LSG-AuNS aptasensor 100 incubated in solutions of various Her-2 protein concentrations from 0.1 ng/mL to 200 ng/mL. Both the sigmoidal curve fitting and logarithmic methods can be used to fit this type of data.

As shown in FIG. 12B inset, a linear-logarithmic relationship was obtained, and the electrochemical response was increased with the increase in the concentration of Her-2 following the regression equation: Δlox=26.6 log C_{Her-}2+71.9 with a correlation coefficient of 0.996. The limit of detection (LOD) was calculated to be 0.008 ng/mL using the above equation by defining the log C_{Her-2} equivalent to the average Alox of the blank plus three times its standard deviation.

The electrochemical response of the LSG-AuNS-MIP sensor 400 was also investigated with respect to the detection of different concentrations of the Her-2. The concentration range tested was from 1 ng/mL to 200 ng/mL. The selection of the concentration range was in agreement with the positive and negative Her-2 level values in the breast cancer patients and healthy samples. As expected, as much as the concentration of the Her-2 was increased, the response of the LSG-AuNS-MIP decreases in the solution of the redox probe of [Fe(CN)₆]^{3-/4-}. This current decrease is due to the binding of Her-2 by the imprinted cavities of the LSG-AuNS-MIP electrode 404, as shown in FIG. 12C.

FIG. 12D shows the corresponding calibration curve obtained with the LSG-AuNS-MIP electrode 404 incubated with different concentrations of the Her-2. In detail, a logarithmic, linear relationship was fitted for the electrochemical responses obtained for the Her-2 captured by the LSG-AuNS-MIP sensor 400 following the equation Δlox=48.04 log [Her-2]+24.96, R2=0.992. As a result, the developed sensor exhibited a LOD of 0.43 ng/ml for the Her-2 detection. These results confirmed that the gold nanostructured LSG electrode could be an excellent candidate in sensing these types of biomarkers due to their easiness of preparation, and practicality when combined with high selectivity and sensitivity of the MIP technology.

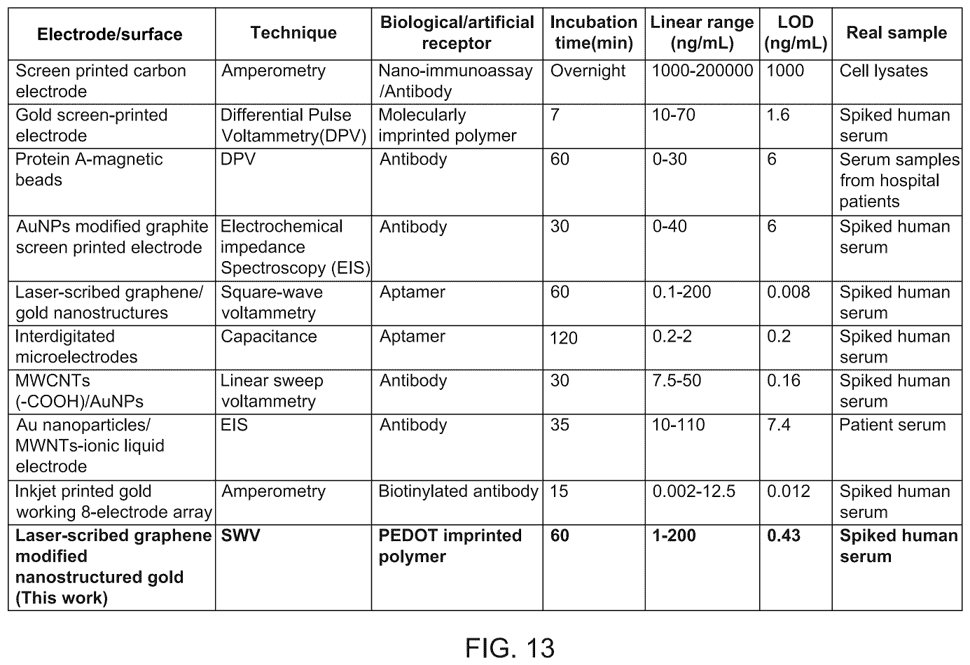

As can be seen in the table of FIG. 13, different Her-2 biosensing approaches have been reported. The reported studies demonstrated the ability to detect Her-2 in real samples below the cut-off concentration value (15 ng/ml), confirming their useful clinical diagnosis application. The developed strategy discussed in these embodiments, based on LSG-AuNS-MIP sensing, confirmed its capability of successfully competing with other bioassay sensing systems to detect the Her-2. Both the LSG-AuNS sensor 100 and the MIP-based sensor 400 proved their low-cost, high sensitivity, and selectivity towards the detection of the Her-2.

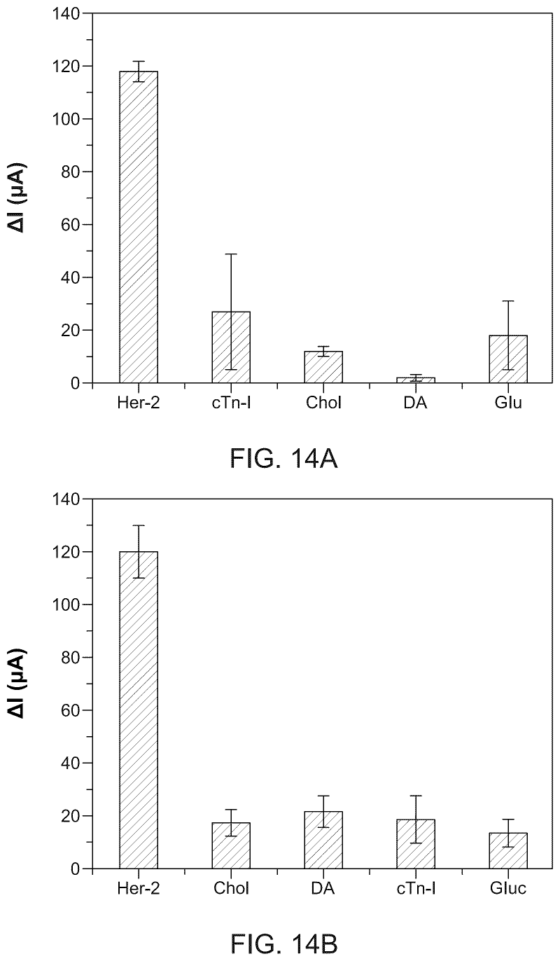

The interference of other possible biomolecules with the sensors 100/400 was studied as now discussed. The affinity of the aptasensor 100 was evaluated in the presence of some possible interferences, including Glucose (Glu), Cardiac troponin I (cTn-I), Cholesterol (Chol), and Dopamine (DA). The amounts of all interferences and the Her-2 were fixed at 50 ng/mL. As shown in FIG. 14A, the proposed aptasensor exhibited high selectivity for the binding of Her-2 due to its high affinity to the aptamer. Significantly, low responses were obtained for Glu, Chol, and DA except for cTn-I, which is about 20% of the response of Her-2 with a large SD value. Among the interference molecules studied, the cTn-I is a protein just like Her-2 with a different amino acid sequence and therefore has a higher tendency for non-specific attachment on the aptasensor surface. In this embodiment, a well-known MCH 412 surface chemistry was used and a Bovine Serum Albumin (BSA)] 414 blocking strategy to reduce the non-specific adsorption. However, it was reported that changing the surface chemistry to sulfobetaine terminated thiol instead of MCH resulted in better antifouling properties of the aptasensor. Nonetheless, the LSG-AuNS-aptasensor 100 showed good selectivity towards detecting Her-2.

The selectivity of the developed LSG-AuNS-MIP sensor 400 was also studied in the presence of the cardiac troponin I (cTn-I), glucose (Gluc), dopamine (DA), and cholesterol (Chol) as show in FIG. 14B. The concentrations of the tested interferences and Her-2 were fixed at 50 ng/ml. The proposed LSG-AuNS-MIP sensor 400 exhibited high selectivity for the binding of Her-2 due to the good selectivity of the MIP matrix 411 towards the analyte. On the contrary, significantly low responses were obtained for other possible interferences due to low interactions and less affinity of the MIP towards these molecules, as also shown in FIG. 14B. These results confirmed the high selectivity of the LSG-AuNS-MIP sensor 400 for the binding of Her-2.

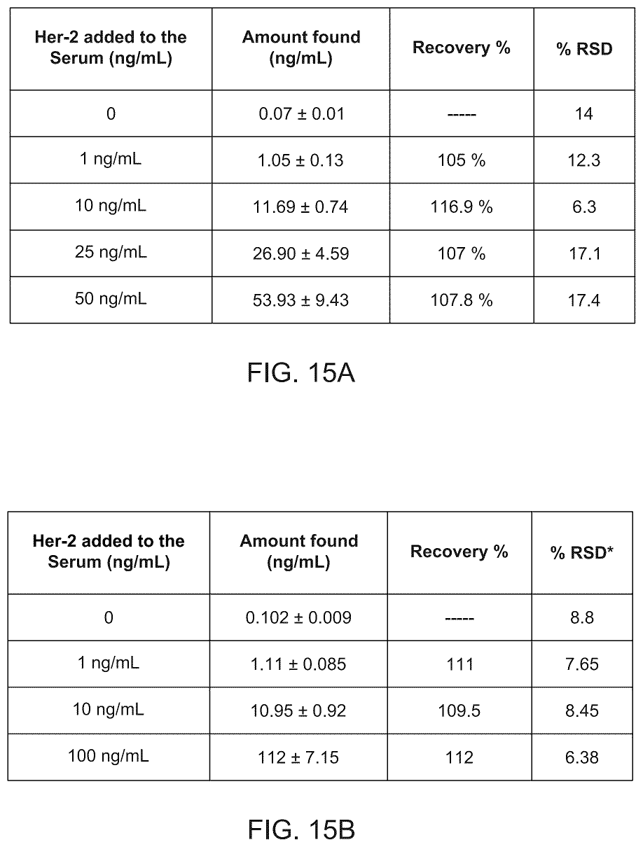

To demonstrate the potential of the LSG-AuNS aptasensor 100 for a real sample, the inventors tested different amounts of Her-2 added to undiluted human serum. The clinically relevant cut-off value for Her-2 is 15 ng/mL. A value above 15 ng/ml is considered as being indicative of Her-2 positive and indicates tumor progression. Hence amounts of 0, 1, 10, 25, and 50 ng/ml of Her-2 protein were added to pure serum samples to determine the recovery values using the SWV technique. As indicated in the table shown in FIG. 15A, for undiluted serum (0 ng/ml spiked Her-2), the recovered value was 0.07+/−0.01 ng/mL, which may be attributed to the presence of intrinsic Her-2 and non-specific adsorption from serum proteins present in the serum sample used in these studies. A similar effect on the aptasensor signal was also observed in the art for detecting Her-2 in the undiluted human serum sample. The percentage recovery values for 1, 10, 25 and 50 ng/ml were 105, 116.9, 107 and 107.8%, respectively, as indicated in FIG. 15A. The higher recovery values may be attributed to intrinsic Her-2 molecules in the undiluted serum and partly due to the non-specific adsorption of other molecules present in the serum sample. It is important to note that overall, the % RSD values become higher in undiluted serum samples including the Her-2 compared to % RSD values for PBS. Indeed, the obtained results demonstrate that the LSG-AuNS aptasensor 100 could easily discriminate between Her-2 positive and Her-2 negative in actual human serum samples. These results show the potential of the developed aptasensor for the detection of Her-2 in patient samples.

To prove the application of the developed LSG-AuNS-MIP sensor 400 to detect the Her-2 biomarkers in the real sample application, different concentrations of Her-2 were added to the undiluted human serum samples. Since the clinically cut-off concentration for Her-2 is 15 ng/ml, the serum samples were spiked with 0, 1, 10, and 100 ng/ml of Her-2 to determine the recovery values. As indicated in the table of FIG. 15B, the recovery value obtained for undiluted serum (0 ng/mL spiked Her-2), was 0.102+0.009 ng/mL, which could be due to the contribution from the intrinsic concentration of Her-2 in the serum sample and non-specific adsorption of serum proteins on the sensor surface. Other studies have also observed a similar sensor response due to the undiluted serum sample for HER-2 detection. The percentage recovery values for 1, 10 and 100 ng/ml were 111, 109.5, and 112%, respectively, with satisfactory % RSD values. These higher recovery values are possibly due to the intrinsic Her-2 protein present in undiluted serum and other interferences that could be adsorbed by non-specific adsorption onto the developed sensor.

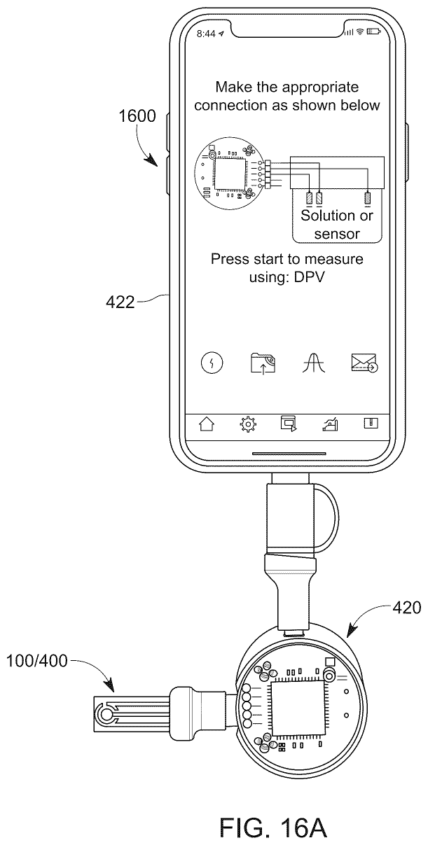

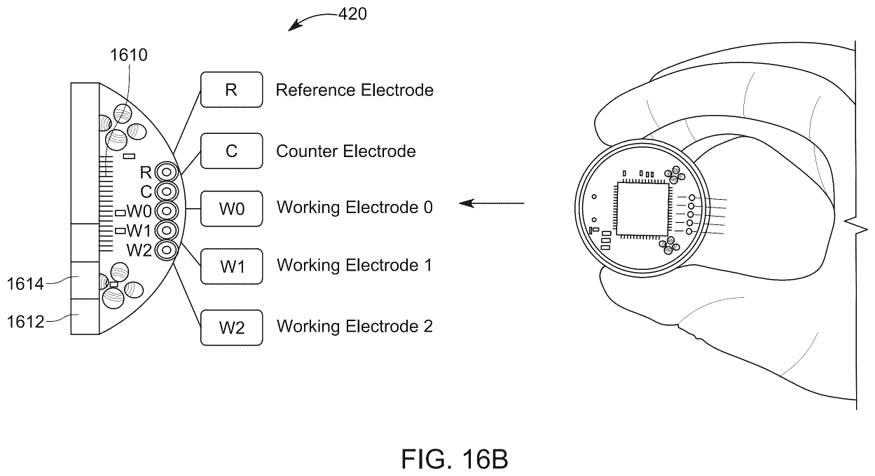

The aptasensor 100 including the LSG-AuNS electrode 104 and the LSG-AuNS-MIP sensor 400 including the LSG-AuNS-MIP electrode 404 have been integrated into a POC system 1600, as illustrated in FIGS. 16A and 16B. The system 1600 includes, in addition to the sensor 100 or 400, the electrochemical analyzer 420, and a smartphone 422. The smartphone 422 uses a mobile application software for analyzing the measurements from the sensor and determining whether the Her-2 protein is above or below a given threshold. An earlier version of the mobile application software was described in Ahmad et al., 2019, KAUST at: a wireless, wearable, open-source potentiostat for electrochemical measurements. IEEE Sens. 19278454. FIG. 16B shows the details of the electrochemical analyzer 420 having a processor 1610, wireless communication unit 1612, various LEDS 1614 for signaling, reference electrode pad R, counter electrode pad C, working electrode pad W0, working electrode 1 pad W1, and working electrode 2 pad W2.

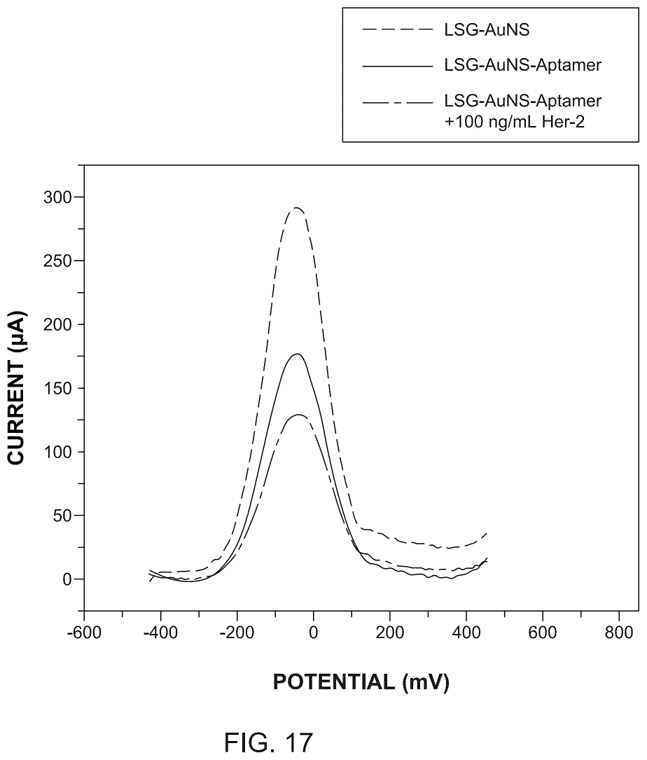

Note that the pads R, C and WO are configured to directly connect to the corresponding pads of the sensor 100/400 and receive their measurements. The analyzer 420 further includes an interface (not shown) for connecting to the smartphone 422. As shown in FIG. 17, the SWVs indicate that the LSG-AuNS electrode 104 or the LSG-AuNS-MIP electrode 404 integrated with the POC device can detect the presence of the Her-2 protein in the sample when compared to the control sample, as there is a clear peak for the current versus voltage curves. The peaks of these curves are indicative of the actual value of Her-2 protein in the sample.

The embodiments discussed above disclose a highly sensitive electrochemical bio-sensing system 100/400/1600, which is based on 3D-porous LSG electrodes modified with 3D gold nanostructures 110. The 3D gold nanostructures are not simply Au particles having a spherical shape. The 3D gold nanostructures 110 have an extended structure, looking like a Christmas-tree, i.e., having a longitudinal axis along the trunk of the tree, and many branches extending away from the trunk. The branches are longer when closer to the surface 104A of the electrode 104, and they grow shorter as they are farther away from the surface 104A. Many Au particles are involved in forming the AuNS 110 while the entire structure still has at least one nanosized dimension (e.g., the thickness of the tree).

In one embodiment, the nanostructures 110 are covered with a polymer 411. The obtained results show a robust method to produce LSG based electrochemical system with better surface coverage, higher sensitivity, and ease of surface modification. The developed sensors allowed sensitive and selective detection of the Her-2 protein in various human serum samples with satisfactory recoveries. An LSG-AuNS sensing system 1600 integrated with a POC device that can be implemented to detect various disease biomarkers has been shown. The sensors 100/400 showed some non-specific adsorption of proteins and an increase in the % RSD values in serum samples that can be improved by employing a better antifouling surface to block non-specific adsorption.

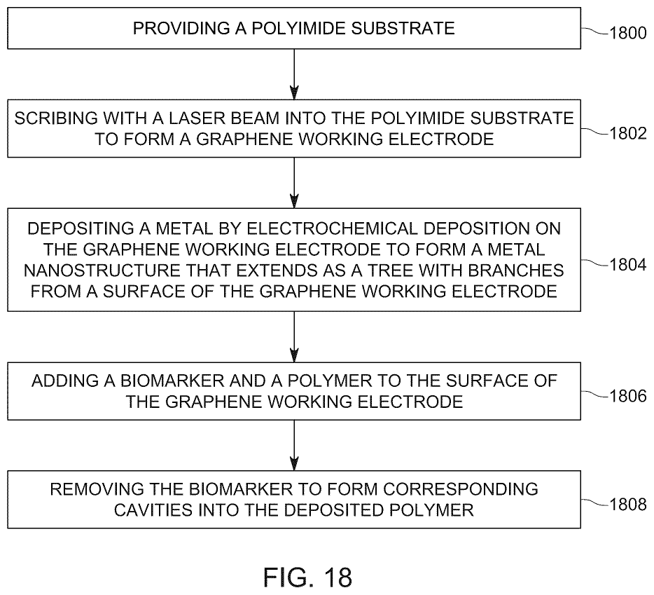

A method for using the system 1600 is now discussed with regard to FIG. 18. The method includes a step 1800 of providing a polyimide substrate, a step 1802 of scribing with a laser beam into the polyimide substrate to form a graphene working electrode, a step 1804 of depositing a metal by electrochemical deposition on the graphene working electrode to form a metal nanostructure that extends as a tree with branches from a surface of the graphene working electrode, a step 1806 of adding a biomarker and a polymer to the surface of the graphene working electrode, and a step 1808 of removing the biomarker to form corresponding cavities into the deposited polymer. Then, the as prepared system can be exposed to the biomarker as shown in FIG. 4K, and a voltage is applied between the electrodes 104 to 108 of the sensor 100/400. The modulated current (signal) is then provided to the analyzer 420, which extracts a signal indicative of the presence of the biomarker. This signal is then sent to the movable computing device 422 to do further processing and display on a screen the value of the biomarker.

The disclosed embodiments provide a laser-scribed graphene sensor having metal nanostructures and an aptamer or molecularly imprinted polymer. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

The entire content of all the publications listed herein is incorporated by reference in this patent application.

Dallinger, A., Keller, K., Fitzek, H., Greco, F., 2020. Stretchable and Skin-Conformable Conductors Based on Polyurethane/Laser-Induced Graphene. Acs Appl Mater Inter 12(17), 19855-19865.

Lahcen, A.A., Rauf, S., Beduk, T., Durmus, C., Aljedaibi, A., Timur, S., Alshareef, H.N., Amine, A., Wolfbeis, O.S., Salama, K.N., 2020. Electrochemical sensors and biosensors using laser-derived graphene: A comprehensive review. Biosens Bioelectron 168, 112565.

Kurra, N., Jiang, Q., Nayak, P., Alshareef, H.N., 2019. Laser-derived graphene: a three-dimensional printed graphene electrode and its emerging applications. Nano Today 24, 81-102.

Lin, X.N., Lu, Z.W., Dai, W.L., Liu, B.C., Zhang, Y.X., Li, J.Y., Ye, J.S., 2018b. Laser engraved nitrogen-doped graphene sensor for the simultaneous determination of Cd (II) and Pb(II). J. Electroanal. Chem. 828, 41-49.

Nayak, P., Kurra, N., Xia, C., Alshareef, H.N., 2016. Highly efficient laser scribed graphene electrodes for on-chip electrochemical sensing applications. Adv. Electron Mater. 2 (10), 1600185.

Y.C. Yu, P.C. Joshi, J. Wu, A.M. Hu Laser-induced carbon-based smart flexible sensor array for multiflavors detection ACS Appl. Mater. Inter., 10 (40) (2018), pp. 34005-34012.

Z.H. You, Q.M. Qiu, H.Y. Chen, Y.Y. Feng, X. Wang, Y.X. Wang, Y.B. Ying, Laser-induced noble metal nanoparticle-graphene composites enabled flexible ionsensor for pathogen detection Biosens. Bioelectron., 150 (2020), p. 111896

G. Selvolini, G. Marrazza, MIP-Based Sensors: Promising new tools for cancer biomarker determination, Sensors-Basel 17 (2017) 718.

Besant, J.D., Das, J., Burgess, I.B., Liu, W.H., Sargent, E.H., Kelley, S.O., 2015, Ultrasensitive visual read-out of nucleic acids using electrocatalytic fluid displacement. Nat. Commun. 6, 6978.

Mahshid, S., Mepham, A.H., Mahshid, S.S., Burgess, I.B., Safaei, T.S., Sargent, E.H., Kelley, S.O., 2016. Mechanistic Control of the growth of three-dimensional gold sensors. J. Phys. Chem. C 120 (37), 21123-21132.

Shu, H.H., Cao, L.L., Chang, G., He, H.P., Zhang, Y.T., He, Y.B., 2014. Direct electrodeposition of gold nanostructures onto glassy carbon electrodes for non-enzymatic detection of glucose. Electrochim. Acta 132, 524-532.

Soleymani, L., Fang, Z.C., Lam, B., Bin, X.M., Vasilyeva, E., Ross, A.J., Sargent, E.H., Kelley, S.O., 2011. Hierarchical nanotextured microelectrodes overcome the molecular transport barrier to achieve rapid, direct bacterial detection. ACS Nano 5 (4), 3360-3366.

S. Rauf, A.A. Lahcen, A. Aljedaibi, T. Beduk, J. Ilton de Oliveira Filho, K.N. Salama, Gold nanostructured laser-scribed graphene: A new electrochemical biosensing platform for potential point-of-care testing of disease biomarkers, Biosensors and Bioelectronics, 180 (2021), pp. 113-116.

1-6. (canceled)

7. A biomarker detection sensor comprising:
a substrate;
a working electrode formed by laser-scribing directly into the substrate so that a material of the substrate is transformed into graphene;
a metal nanostructure formed on a graphene surface of the working electrode, wherein the metal nanostructure is shaped as a tree with plural branches extending away from the graphene surface;
a polymer covering the graphene surface of the metal nanostructure, wherein plural cavities are formed in the polymer with a biomarker to be detected;
a reference electrode; and
a counter electrode.

8. The sensor of claim 7, wherein the polymer also covers the graphene surface.

9. The sensor of claim 7, where each of the plural cavities is shaped and size to accept only the biomarker.

10. The sensor of claim 7, wherein the metal nanostructure is gold, and the polymer is 3,4-ethylenedioxythiophone or poly-3,4-ethylenedioxythiophone.

11. The sensor of claim 10, wherein the biomarker is a Her-2 protein.

12. The sensor of claim 11, wherein each of the plural cavities is shaped by the Her-2 protein.

13. The sensor of claim 7, wherein the graphene surface of the working electrode covered by the metal nanostructure is circular.

14. A method for making a biomarker detection sensor, the method comprising:
providing a polyimide substrate;
scribing with a laser beam into the polyimide substrate to form a graphene working electrode;
depositing a metal by electrochemical deposition on the graphene working electrode to form a metal nanostructure that extends as a tree with branches from a surface of the graphene working electrode;
adding a biomarker and a polymer to the surface of the graphene working electrode; and
removing the biomarker to form corresponding cavities into the deposited polymer.

15. A system for determining a biomarker, the system comprising:
a biomarker detection sensor;
a signal analyzer configured to directly connect to the biomarker detection sensor to receive measurements and generate a signal indicative of the biomarker; and
a portable computing device that receives the signal and displays the signal on a screen,
wherein the biomarker detection sensor comprises:
a working electrode formed by laser-scribing directly into a substrate so that a material of the substrate is transformed into graphene,
a metal nanostructure formed on a graphene surface of the working electrode, wherein the metal nanostructure is shaped as a tree with plural branches extending away from the graphene surface, and
a polymer covering the graphene surface of the metal nanostructure, wherein plural cavities are formed in the polymer with a biomarker to be detected.

16. The system of claim 15, wherein the polymer also covers the graphene surface.

17. The system of claim 15, wherein each of the plural cavities is shaped and sized to accept only the biomarker.

18. The system of claim 15, wherein the metal nanostructure is gold, and the polymer is 3,4-ethylenedioxythiophone or poly-3,4-ethylenedioxythiophone.

19. The system of claim 18, wherein the biomarker is a Her-2 protein.

20. The sensor of claim 18, wherein each of the plural cavities is shaped by the Her-2 protein.
